(19) United States
(12) Patent Application Publication
(10) Pub. No.: US 2016/0051248 A1
(43) Pub. Date: Feb. 25, 2016

(54) SURGICAL NEEDLE HAVING A DETACHABLE TIP BODY AND A THREAD RUNNING INSIDE

(71) Applicant: DOCI INNOVATIONS GMBH, Sierksdorf (DE)

(72) Inventor: VIOLETA DOCI, Hamburg (DE)

(21) Appl. No.: 14/888,294

(22) PCT Filed: May 5, 2014
(86) PCT No.: PCT/EP2014/059141

§ 371 Date: Nov. 5, 2015

(30) Foreign Application Priority Data
May 5, 2013 (EP) 13166569.7

(51) Int. Cl. A61B 17/04

(52) U.S. Cl. CPC A61B 17/0401; A61B 2017/00792

(57) ABSTRACT

The present invention relates to a surgical needle, which can be used in a medical application for suturing wounds. The present invention more specifically relates to a surgical needle that is connected to a thread and has a tip body, which carries the needle tip, and a needle body, wherein the needle body is helical and has an inner hollow space, and the needle body has a first end, which faces towards the tip body, and the needle body has a second end, which is opposite the first end, and the tip body has a thread receptacle and the thread is guided from the thread receptacle from the first end of the needle body through the hollow space to the second end of the needle body, characterised in that the tip body is formed as a separate component, which is detachably connected to the needle body, and has a tip body, shank piece and connection piece and is rotationally symmetrical about an axis through the needle tip.

### FIELD OF THE INVENTION

The present invention relates to a surgical needle that can be used to suture wounds in a medical application.

### BACKGROUND OF THE INVENTION

The suturing of wounds in order to initiate wound healing is an old technique. Accordingly, there are a large number of variants for different applications. The typical hand tools for doctors are still a needle and thread. Both are available in a broad selection and can be matched to the respective application. Finished needle/thread combinations are also typically available on the market.

European patent EP 1 726 317 B1 discloses surgical means and a surgical method suitable in particular for cosmetic operations. Here, a thread is guided into the tissue in a plurality of spirals, approximately in the form of a helix overall. This is achieved using a straight needle.

European patent EP 1 778 096 B1 discloses a spiraled suture device. This suture device is intended especially for applications in the intervertebral disc area. The device has a needle in the form of a helix. The needle is internally hollow and may guide a thread in the hollow space. The thread is guided as a loop at the end of the helical needle, i.e. in the vicinity of the tip of the needle. It is possible using an instrument having a grab hook (not dissimilar to a hook needle) to engage the loop of the thread at the front end of the helical needle and to then grasp this thread. The thread can thus be guided over a helical path and then knotted. What is clearly demanding in the method is that two instruments are required and the thread must be brought into a position where it can be easily grasped by the hook.

U.S. Pat. No. 5,356,424 discloses a needle/thread combination especially for minimally invasive surgery. Here, a needle is used that likewise has a helical portion. This needle is internally hollow and guides a thread. The needle sits on a needle holder, which for example has the form of a pin. The needle is guided in this needle holder. The needle, however, is detachably connected to the needle holder and may also be removed. It should be noted that the needle holder has a design matched especially to the needle, this design deviating significantly from the construction of conventional approximately forceps-shaped needle holders. One type of application of this apparatus lies in pulling the thread from the needle at the pointed end. It is clear that the thread is difficult to guide here and may easily break.

The German unexamined application DE 10 2008 044 855 discloses an apparatus and a method for closing wounds by surgery. Here, a hollow spiral needle is used. In the vicinity of the tip the spiral needle has a predetermined breaking point, at which the tip can be separated from the rest of the needle body. The tip may then be securely held using surgical forceps.

In the field of threads there are a large number of developments and improvements. Modern threads formed from a plurality of components are disclosed for example by European patent specification EP 1 171 172 B1 and also European patent specification EP 1 293 218 B1, which disclose a supposedly particularly strong thread material.

The present invention seeks to improve this prior art. In particular, a surgical needle is to be provided with which sutures can be reliably made, even in areas that are difficult to access. Here, the suture is to be made easily and the thread is to run in an easily predeterminable manner. The return of the needle from the tissue is to be easy and reliable and the correct placement of the thread as it is returned is to be ensured even in difficult situations.

### DETAILED DESCRIPTION OF THE INVENTION

This object is achieved by a surgical needle according to claim 1 and correspondingly by a method according to claim 13.

The present invention thus relates to a surgical needle which, in accordance with claim 1, has a tip body and a needle body as key elements. The tip body carries the needle tip, i.e. is positioned at the pointed end of the needle. The tip body itself has a shank piece adjoining the needle tip and a connection piece. The connection to the needle body can be produced by means of this connection piece.

The needle body itself is generally the main part of the needle in terms of weight and size. It is helical in the context of the present invention. Here, this is not a mathematically exact helix, but rather the needle body is round or approximately circular or is elliptical or approximately elliptical in a projection plane and is formed in a plane perpendicular thereto such that the needle is advanced by rotation in the tissue. A suitable form in this case is a zigzag form with three-dimensional extension. However, a mathematically exact or approximated mathematically exact helix is absolutely expedient.

The helical needle body has an inner hollow space. A thread may be guided in this inner hollow space. The hollow space has a first end, which faces towards the tip body and therefore the needle tip. The hollow space also has a second end, which is opposite the first end. The thread may protrude beyond the second end.

In order to guide the thread, the tip body has a thread receptacle at the first end, i.e. in the vicinity of the first end of the hollow body. The form of the thread receptacle is not very decisive. In principle, a conventional needle eye could be considered, however a bead is also possible, into which a thread end is merely inserted. The thread may also be adhered to a guide surface or wound around part of the tip body. The surgical needle is generally delivered ready with a thread. The thread receptacle may therefore be designed such that it is suitable only for one thread.

In the context of the present invention the tip body is formed as a separate component. It is to be connected detachably to the needle body. The tip body is to have a needle tip, a shank piece and a connection piece. This connection piece serves to connect the needle body. Furthermore, the tip body is to be rotationally symmetrical about an axis through the needle tip.

It has been found that the rotationally symmetrical embodiment is helpful, particularly when treating very small wounds. It is helpful in particular in combination with the helical needle body. Due to the helix form of the needle body, pressure is not exerted directly onto the needle tip. The pressure is created substantially by a rotary movement at the other end of the helix, i.e., by contrast with a straight needle, there is no straight line from the point at which pressure is exerted to the needle tip. In addition, it may be easy for the helix to warp under pressure, said helix being formed as a hollow space and often as a thin-walled hollow space.

Under these circumstances it would be easily conceivable in principle for the needle tip to assume an angle that is difficult to predict relative to the tissue, and the pressure point also is not ideal. The penetration of the tissue may thus be more difficult than necessary, and more damage than is actually necessary may be caused in the tissue.

A typical simple needle tip is presented for example in European patent EP 1 778 096 81, which has already been cited. There, a typical tip form is presented as produced by a mere inclined cut through a tube end. As mentioned, this tip form is not ideal, particularly when placing a surgical suture of very small dimension, for example in the case of an eye operation.

The rotationally symmetrical embodiment of the shank piece about an axis through the needle tip ensures that the shank piece can effectively take up pressure and releases this at the needle tip in a predictable and controlled manner. Here, no large transverse forces are created, such that the needle tip remains on its target path.

It is expedient when a bearing is provided on the needle body, by means of which bearing it is possible for pressure to be transmitted to the tip body. Such a bearing causes a defined pressure transmission. In particular, it is expedient when the bearing has a peripheral support face. A peripheral support face may run here around the axis through the needle tip over 360 degrees. It may also be sufficient to provide a peripheral, but interrupted support face, which for example covers only three 100° segments or four 60° segments, or the like, running around the axis and around the needle tip. The support face here may be an end face (for example a cut face) or a protrusion.

It is also advantageous when a form-fitting lateral guide is provided in the needle body for the connection piece of the tip piece. Such a form-fitting lateral guide prevents the canting of the tip piece relative to the needle body. By way of example, the connection piece may be formed as a tube piece and the needle body may end with a tube piece of greater circumference, which receives the connection piece with an accurate fit. A form-fitting lateral guide makes it possible in a simple manner to make the tip body detachable from the needle body.

It is also expedient when the tip body transitions flush into the needle body. In this way a smooth transition between tip and needle is provided at the outer surface. Unnecessary damage to the tissue is thus avoided.

A stop may also be provided on the tip body. This stop serves to initiate the detachment of the tip body from the needle body. Such a stop is to form a resistance when the needle is moved backwards. At least one fold-out pin is expedient for this purpose. The stop initiates the detachment of the tip body from the needle body, but does not serve as the only resistance against a backwards movement of the tip body. Rather, a further component may be provided for this purpose, for example a spreading fan, which is effective following detachment of the tip body from the needle body.

It may therefore also be particularly advantageous when a spreading fan is provided on the tip body. This spreading fan prevents a thread from sliding back when the needle is guided backwards. The function of this fan will become clearer from the comments relating to the method for placing a surgical suture and from the drawings. It is then also advantageous when a receiving space for the spreading fan is provided in the needle body.

It should be noted that the provision of a spreading fan on the tip body is fundamentally advantageous and for example may also be desired, irrespective of the geometry of the tip body. The invention therefore also relates to a surgical needle connected to a thread and having a tip body, which carries the needle tip, and a needle body, wherein the needle body is helical and has an inner hollow cavity, and the needle body has a first end facing towards the tip body, and the needle body has a second end opposite the first end, and the tip body has a thread receptacle and the thread is guided from the thread receptacle from the first end of the needle body through the hollow space to the second end of the needle body, wherein the tip body is formed as a separate component, which is detachably connected to the needle body, and has a tip body, shank piece and connection piece, and a spreading fan is provided on the tip body.

The spreading fan preferably comprises a number of lamellae that is not too small and that is preferably even, since it has been found that the spreading fan can then be removed more reliably from the receiving space and is supported predictably and uniformly. Three, but more preferably (since even) four or more lamellae are advantageous. The lamellae are preferably spring-mounted in a preloaded manner in the receiving space.

The lamellae are expediently manufactured from metal or plastic. Since the support of the needle tips by the lamellae may lead to irritation or even injury of very sensitive tissue when the needle body is returned, antimicrobial materials are very advantageous. Such materials may release biocides. By way of example, polypropylene (PP) or polyoxymethylene (POM) may be used. Corresponding antimicrobial qualities are sold inter alia as TECAPRO SAN (PP) and TECAFORM AH SAN (POM-C) (protected trademarks). The individual lamellae may advantageously have a rectangular flat profile.

Here, it is advantageous if the lamellae can spread open widely. The ends of the lamellae advantageously describe a supporting diameter that is greater than the outer diameter of the needle body at least by a factor of 3, more preferably a factor of 5 or 10. (Here, the greatest distance between two substantially opposed lamella ends of the supporting diameter). The broad support is gentle on the tissue and prevents a tilting of the needle tip. Furthermore, the greatest angular range possible is to be covered by the lamella ends (in relation to the 360° angle of a full circle). A supporting range of more than 20°, 30°, 40° or 60° is advantageous, wherein 90° must not be exceeded. This measure is also gentle on the tissue and prevents a tilting of the needle tip.

When making very small surgical sutures, there is a preference for smooth threads. Such smooth threads cause minimal damage to tissue. A corresponding preference for mono-filament threads is also ascertained in the German unexamined publication DE 10 2008 044 855. However, it has surprisingly been found within the scope of the present invention that a multi-fibre thread is also very expedient. A thread having a rough surface is absolutely advantageous. The multi-fibre nature of the thread and in particular the resultant rough surface specifically lead to a low frictional resistance between the thread and the surgical needle. This makes it particularly easy to return the surgical needle. Here, a particularly expedient thread is an interwoven or spun thread.

A surgical needle in which the tip can be detached from the needle body by tensile forces of less than 1 Newton (N) or even less than 0.5 N or less than 0.25 N is advantageous.

It is important to match the type and size of the spreading fan to the type of thread. In principle, a surgical needle that in combination has a spreading fan and a braided or spun thread is advantageous. The structure of the thread allows the thread to be removed from the needle body under application of forces signifying a moderate loading of the spreading fan and the tissue against which the spreading fan is supported.

Within the scope of the present invention it has been found that a method of the following type is useful:

A method for placing a surgical needle between a first wound tissue and a second wound tissue, in which a thread and a surgical needle having a needle body and a detachable tip, at which the thread is held, are used, wherein the method comprises the following steps in the listed order:
puncturing the first wound tissue using the needle
guiding the needle on to the second wound tissue, wherein the needle is guided through the puncture in the first wound tissue
puncturing the second wound tissue, wherein the needle is guided on through the puncture in the first wound tissue
guiding the tip from the second wound tissue, wherein the needle is guided on through the puncture in the first wound tissue, and wherein the path of the tip of the needle in the previous steps defines an insertion direction and an insertion path
detaching the needle tip from the needle body substantially by exerting tensile force against the insertion direction
returning the needle body without the tip from the second wound tissue and from the first wound tissue in the reverse direction along the insertion path, wherein the thread remains in the wound tissue

Such a method is also useful in the more commercial fields of medicine, for example in cosmetic surgery, which also includes interventions on healthy patients without medical necessity.

From the understanding of this method, many advantages of the invention can be particularly clearly identified. It should also be noted that this method can be used for application in humans, but also in veterinary medical applications, i.e. for treating wounds in animals.

The method may be supplemented by a step in which only the needle body is held as the needle body is returned. Furthermore, only the needle tip may be supported with a spreading fan on the wound tissue or on adjacent tissue as the needle body is returned. This method is particularly suitable for wound tissue in areas that are difficult to access.

Thus, one concern of the invention is to provide material for carrying out such a method, in particular a suitable surgical needle.

Further features, but also advantages of the invention will become clear from the drawings discussed hereinafter and the associated description. Features of the invention are described in combination in the figures and in the associated descriptions. However, these features may also be included in other combinations by a subject according to the invention. Each disclosed feature is therefore also to be considered as disclosed in technically feasible combinations with other features. In the (partially slightly simplified, schematic) figures:

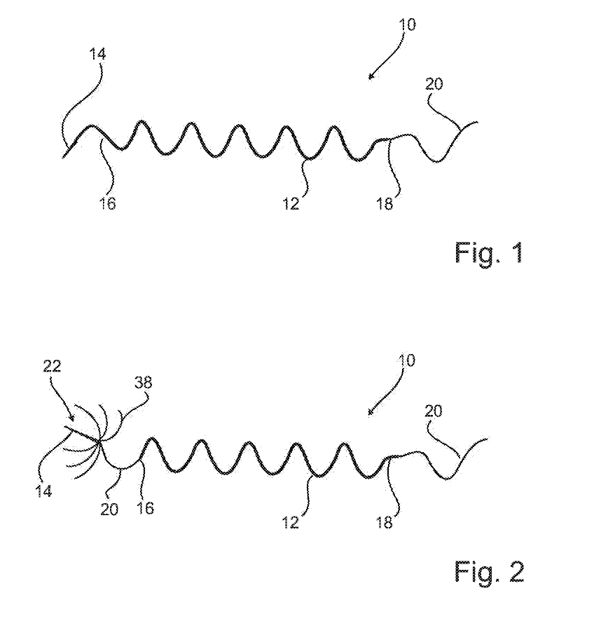

FIG. 1 shows a perspective view of a surgical needle according to the present invention;

FIG. 2 shows a surgical needle according to the present invention, likewise in a perspective view, in which the tip body has been detached from the needle body;

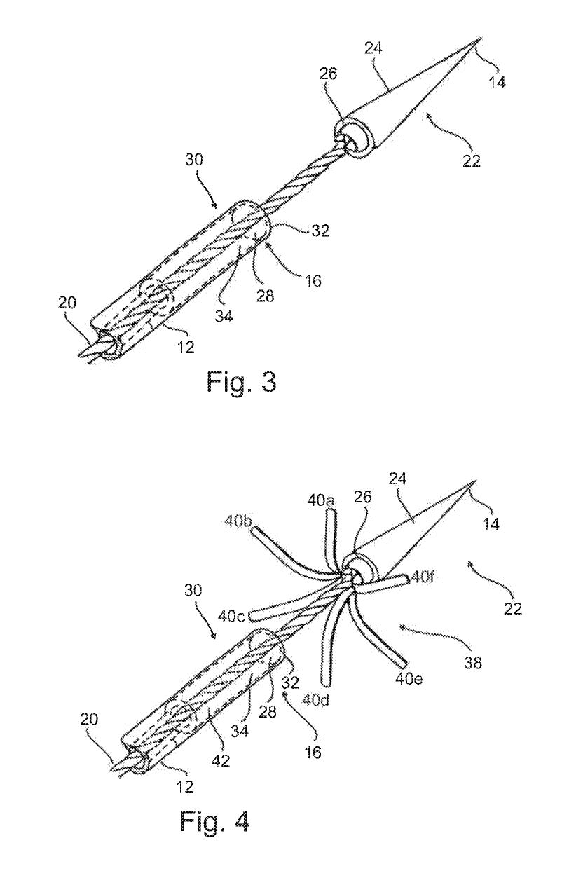

FIG. 3 shows an enlarged view of an end of the needle body and the tip body in accordance with an embodiment of the present invention;

FIG. 4 shows an enlarged view of an end of the needle body and the tip body in an embodiment with a spreading fan, such that the enlarged view corresponds to the embodiment from FIG. 2.

FIG. 1 shows the surgical needle 10 in a (representationally slightly simplified) perspective view. The surgical needle 10 shows the needle body 12 as key component. This needle body has the form of a helix. The needle tip 14 is formed at one end. The first end 16 of the needle body 12 is located directly adjacently of the needle tip 14. The second end 18 of the needle body 12 lies opposite said first end. The thread 20 protrudes from the second end 18.

The surgical needle 10 is thus shown here in the state prior to or during the suturing of a wound. The needle tip 14 (which sits on a tip body not visible here) is fixedly connected to the needle body 12 in this state.

FIG. 2 shows, in a corresponding view, a surgical needle 10 according to the invention in another state of use. Again, the needle body 12 of the surgical needle 10 and the first and 16 thereof and the second end 18 thereof can be seen. However, the tip body 22 is detached from the needle body 12. The thread 20 is still connected to the first end 16 of the needle body 12 between the tip body 22. In this embodiment the tip body 22 has a spreading fan 38. As described, the spreading fan 38 can be supported on a tissue part (more specifically generally without damaging this in any way). The return of the needle body 12 (substantially) over the insertion path, but in the opposite direction and therefore along a return path ensures that the needle body 12 and the tip body 22 are distanced further and further from one another. Both are still connected by the thread 20. The thread 20 during this movement slides through the hollow cavity in the needle body 12.

The length of the thread 20, which still protrudes beyond the second end 18 of the needle body 12, reduces accordingly.

FIG. 3 in an enlarged view shows a tip body 22 and the needle body 12 in the region of its first end 18. FIG. 3 shows these components for a needle according to the present invention, but in an embodiment different from that shown in FIG. 2. As can be clearly seen in this representation, the tip body 22 has the needle tip 14 at the front and a connection piece 26 facing the needle body 12. Between the connection piece 26 and needle tip 14, the tip body 22 has a shank piece 24. It can be seen that the tip body 22 is rotationally symmetrical about a main axis through the needle tip 14.

The tip body 22 is connected to the thread 20 in a manner not illustrated in greater detail. The thread is a multi-fibre thread. This thread 20 runs in the hollow cavity in the needle body 12, which is illustrated here only in the region of its first end 16. In the region of the first end 16, the hollow cavity in the needle body 12 is widened. This can be achieved for example by drilling.

In this region a bearing 30 is formed, which comprises a support face 32 and a lateral guide 34. An end face, which is smooth and constitutes a support face 32, is formed at the first end 16 of the needle body 12. This support face 32 runs around the longitudinal axis of the needle body 12 in this end region. It thus describes a 360-degree angle around the thread 20.

A receiving space 28 for the connection piece 26 is also provided at the first end 16. The tubular design of the needle body 12 in this region already also ensures that there is a lateral guide 34 for the connection piece 26. The support face 32 and the lateral guide 34 in this way here offer on the whole a bearing 30, in which the tip body 22 can be mounted, as shown preferably via its connection piece 26.

FIG. 4 in the same view as FIG. 3 shows another embodiment of the needle tip 14. The basic elements that have been described for FIG. 3 are also used in this embodiment. However, this embodiment additionally has a spreading fan 38. This spreading fan 38 consists of six spreading lamellae 40a, 40b, 40c, 40d, 40e and 40f. These lamellae are fixedly connected to the tip body 22. It can also be seen in this figure that the receiving space 28 is longer than is necessary in order to receive the connection piece 26. The receiving space 28 is therefore adjoined by a receiving space 42, which can accommodate the spreading lamellae 40. Prior to the separation of the tip body 22 from the needle body 12, the lamellae lie in the receiving space 42. This makes it possible in the first phase of the use of the needle for the needle body to transition flush at its first end 16 into the tip body 22. The spreading lamellae 40 are then completely hidden in the receiving space 42. Since the lamellae spread out independently, they press in this state against the inner walls of the receiving space 42. This ensures an even more secure connection between the tip body 22 and the needle body 12.

On the whole, it is shown that new possibilities for the improved suturing of wounds are provided with this concept.

### LIST OF REFERENCE SIGNS

10 surgical needle
12 needle body
14 needle tip
16 first end
18 second end
20 thread
22 tip body
24 shank piece
26 connection piece
28 receiving space
30 bearing
32 bearing face/pressure face/contact face
34 side guide
36 stop
38 spreading fan
40 spreading lamellae
42 receiving space for spreading fan

1-15. (canceled)

16. A surgical needle (10), which is connected to a thread (20) and has a tip body (22), which carries the needle tip, and a needle body (12), wherein the needle body (12) is helical and has an inner hollow space, and the needle body (12) has a first end (16), which faces towards the tip body (22), and the needle body (12) has a second end (18), which is opposite the first end, and the tip body (22) has a thread receptacle and the thread (20) is guided from the thread receptacle from the first end of the needle body (12) through the hollow space to the second end of the needle body (12), wherein the tip body (22) is formed as a separate component, which is detachably connected to the needle body (12), and has a tip body (22), shank piece (24) and connection piece (26), characterised in that the tip body (22) is rotationally symmetrical about an axis through the needle tip and a spreading fan (38) is provided on the rotationally symmetrical tip body (22).

17. The surgical needle (10) according to claim 16, in which a bearing (30) is provided on the needle body (12), by means of which bearing it is possible for pressure to be transmitted to the tip body (22).

18. The surgical needle (10) according to claim 17, in which the bearing (30) has a peripheral support face (32).

19. The surgical needle (10) according to claim 16, in which a form-fitting lateral guide (34) is provided for the connection piece (26) of the tip body (22) in the needle body (12).

20. The surgical needle (10) according to claim 16, in which the tip body (22) transitions flush into the needle body (12).

21. The surgical needle (10) according to claim 16, in which a stop is provided on the tip body (22).

22. The surgical needle (10) according to claim 16, in which the spreading fan (38) comprises spreading lamellae (40) preferably four to ten lamellae.

23. The surgical needle (10) according to claim 16, in which a receiving space (42) for the spreading fan (38) is provided in the needle body (12).

24. The surgical needle (10) according to claim 16, in which the thread (20) is a multi-fibre thread.

25. The surgical needle (10) according to claim 24, in which the thread (20) is braided or spun.

26. The surgical needle (10) according to claim 16, in which the tip can be detached from the needle body (12) by tensile forces of 1 N or less.

27. A method for medical, cosmetic surgical, veterinary medical or commercial application, said method comprising the following steps:
a. puncturing the first wound tissue using the needle,
b. guiding the needle on to the second wound tissue, wherein the needle is guided through the puncture in the first wound tissue,
c. puncturing the second wound tissue, wherein the needle is guided on through the puncture in the first wound tissue,
d. guiding the tip from the second wound tissue, wherein the needle is guided on through the puncture in the first wound tissue, and wherein the path of the tip of the needle in the previous steps defines an insertion direction and an insertion path,
e. detaching the needle tip from the needle body (12) substantially by exerting tensile force against the insertion direction,
f. returning the needle body (12) without the tip from the second wound tissue and from the first wound tissue in the reverse direction along the insertion path, wherein the thread (20) remains in the wound tissue.

28. The method according to claim 27, in which the tip is not held as the needle body (12) is returned.

29. The method according to claim 27, in which the tip is supported via a spreading fan (38) on the first wound tissue or on the second wound tissue or on tissue adjacent to the first or the second wound tissue.
